(19) United States
(12) Patent Application Publication
(10) Pub. No.: US 2015/0283095 A1
(43) Pub. Date: Oct. 8, 2015

(54) NANOPARTICLES WITH BIODEGRADABLE AND BIOCOMPATIBLE POLYMER PLGA, LOADED WITH THE DRUG FOR HUMAN USE PENTOXIFYLLINE

(71) Applicant: UNIVERSIDAD DE SANTIAGO DE CHILE, Santiago (CL)

(72) Inventors: Luis Ernerto Constandil Córdova, Santiago (CL); Paula Soledad Ibarra Durán, Santiago (CL); Cristian Andres Vilos Ortiz, Santiago (CL); Luis Velásquez Cumplido, Santiago (CL); Teresa Pelissier Serrano, Santiago (CL); Claudio Aurelio Laurido Fuenzalida, Santiago (CL); Alejandro Hernández Kunstmann, Santiago (CL)

(73) Assignee: UNIVERSIDAD DE SANTIAGO DE CHILE, Santiago (CL)

(21) Appl. No.: 14/443,260

(22) PCT Filed: Nov. 15, 2013
(86) PCT No.: PCT/CL2013/000084

§ 371 Date: May 15, 2015

(30) Foreign Application Priority Data
Nov. 16, 2012 (CL) 3209-2012

(51) Int. Cl. A61K 9/51; A61K 9/00; A61K 9/50; A61K 31/522

(52) U.S. Cl. CPC A61K 9/5153; A61K 31/522; A61K 9/0019; A61K 9/5089

(57) ABSTRACT

The invention relates to a novel pharmaceutical formulation comprising polymer nanoparticles of the biodegradable and biocompatible polymer poly(lactic-glycolic) acid (PLGA), loaded with the drug pentoxifylline, the method for the synthesis of the PLGA nanoparticles loaded with pentoxifylline, and to the use thereof in the effective treatment for the relief of chronic pain and for the prevention of chronic pain via the administration of a single dose.

### FIELD OF THE INVENTION

The present invention relates to pharmaceutical and nanotechnology industry, especially with the use of biodegradable and biocompatible polymer poly(lactic-glycolic acid) (PLGA for its acronym in English “poly(lactic-glycolic) acid)”) and pentoxifylline to generate a new pharmaceutical form (nanoparticle-drug) allowing the reduction to a single dose for effective treatment of chronic pain and to prevent the onset of chronic pain drug.

### BACKGROUND OF THE INVENTION

The “pain” or nociception (named after the Latin word “nocere” meaning damage) is an experienced unpleasant feeling when tissues are injured. This is how the sensation of pain protects the body against an imminent threat or potential injury. For many, the “pain” or nociceptive experience has been temporary (or acute). However, for others the experience is endless, becoming a chronic (or pathological) pain.

According to the International Association for the Study of Pain (IASP, for its acronym in English “international Association for the Study of Pain”), acute pain is one that remains the time necessary to recover the damaged tissues. This type of pain is described as an electric severe pain, throbbing, and disappears when tissue damage has been repaired. But sometimes the pain can last over time, becoming a chronic type (Paeile and Bilbeny, 2005). From the pharmacological point of view and treatment, there are several drugs, including nonsteroidal antiinflammatory drugs (NSAIDs) (ibuprofen, naproxen) or opioids (Demerol, tramadol, oxycodone, etc.) to alleviate acute pain with good efficacy. However in chronic pain, such as that occurs in some cancers, neuralgia, neuropathies and arthritis, its effectiveness (Mantyh et al., 2002; Coutaux et al, 2005) Limited. Therefore, there is no effective drug treatment for chronic pain, affecting the quality of life of people and their families. Today is a public health problem with a prevalence of around 20% of the population.

Several drugs have been tested in pre-clinical research to relieve chronic pain, but have generally shown poor effect (Chou R et al, 2007). One feature of chronic pain is their maintenance in time; therefore therapeutically promising techniques are used for administering analgesics continuously. One approach to the problem is to use slow release drug systems, in order to locally release small amounts of drugs (known analgesics) in prolonged time and thus prevent and/or reverse chronic pain.

Pain and Nociception: Basic Concepts

The pain is usually started at the periphery, mainly by stimulation of free nerve endings. These free nerve endings are stimulated directly by nociceptive stimuli, such as capsaicin (itching substance that gives the pepper), ATP, protons and proteases (released when cells are injured). Furthermore the free ends can be activated by inflammatory mediators such as histamine, bradykinin, nitric oxide and cytokines (Livingston A, 1999; Lipton et al., 1994). When the free ends are activated by the aforementioned stimuli release neuropeptides such as substance P (SP) and related peptide calcitonin gene (CGRP by its acronym “calcitonin gene related peptide”) (Chizh B A, 2002), which act on the same or free ends of those more distant and were not initially activated. This phenomenon where the nervous system is involved in the inflammation processes known as neurogenic inflammation (Richardson and Vasko, 2002).

Whatever form of stimulation of nociceptive nerve terminals, they generate a depolarization of the membrane potential and action leading nociceptive information to the spinal cord. The soma of the first neuron of the nociceptive pathway is located in the dorsal spinal ganglion and its axon is divided into T shape, one branch leading to the body surface and the other to the spinal cord. Axons leading type information are classified into nociceptive A∂ fibers and C fibers. The A∂ fibers are characterized by myelinated and carry the signal between 4 to 30 meters per second, whereas the C fibers are unmyelinated and conduct information between 0.4 to 2 meters per second. Prolongation of nociceptive neuron axon that goes to the dorsal horn of the spinal cord, neuron synapses with a second order of the pain pathway, which projects its axon to higher centers where the evaluation and interpretation occurs nociceptive information.

The sensory, insular and prefrontal cortices are involved in the perception and mediate emotional responses, somatic reflex, reflex autonomic, endocrine, learning and memory (Codere et al, 1993; Scholz and Woolf, 2002)

Synaptic transfer of nociceptive information to the spinal level, is made possible by the release of excitatory mediators such as glutamate, SP, CGRP, nitric oxide, and neurotrophins such as brain-derived neurotrophic factor (BDNF for its acronym in English, “brain derived neurotrophic factor”). Most of these neurotransmitter/neuromodulators released by nociceptors excitatory actions occur in second order neurons from the spinal cord through the opening of cation channels permeable and/or through intracellular signal cascades triggered by activation phospholipase C or adenylyl cyclase and activation of tyrosine kinase receptors. The spinal cord is the first relay point of nociceptive information and modulation of endogenous, meaning that the spinal cord qualifies as a major point of pharmacological modification.

Chronic Pain and Central Sensitization

When a nociceptive sensation extends in time and time exceeds tissue repair, the pain starts to be pathological or chronic. When pain becomes chronic, it is characterized in that there is an increase in the excitability of neurons of nociceptive pathway (spinal and supraspinal), relative to a nociceptive stimulus. This exaggerated response is recognized in behavioral studies as aliodynia (painful response or aversion caused by a non-nociceptive stimulus) and hyperalgesia (exaggerated painful or aversion response caused by nociceptive stimulation). Altered pain perception in stimulus correlates with changes or alterations in neural plastic substrate related nociceptive pathways, which are known as central sensitization as a whole. (Haydon P, 2001). Among the changes that occur in chronic pain they have been described:

1. An increase in glutamate release which binds to receptors N-methyl D-aspartate receptor (NMDAR) and metabotropic glutamate receptors (mGluR), which produces changes in the expression of oncogenes (for example: src abl), protein synthesis (GluR1-5, NK, NR1, NR2A-B, etc.), enzyme activation (protein kinases and clicooxigenasa (COX), among others); allowing neuronal facilitation (Goicoechea and Martin, 2006).

2. An increased COX expression and nitric oxide in the postsynaptic neuron. Both substances are able to diffuse into the presynaptic neuron, where it does NOT stimulates the release of SP, while COX promotes the synthesis of prostaglandin E (PGE) (Turk and Okifuji, 2001).

3. Phosphorylation of proteins such as ion-channel receptors or associated regulatory proteins, or by altering the intrinsic functional expression on the cell surface of the channels, both in primary sensory neurons in dorsal horn neurons properties. A affected intracellular signaling cascades involved in the interactions of serine-threonine and tyrosine kinase (Woolf and Salter, 2000).

4. Changes at the level of dorsal horn neurons, mediated by activation of MAPK-pCREB cascade- (for its acronym in English “mitogen-activated protein kinases—cAMP response element binding protein”). These include changes in the receptors (neurokinin 1 (NK1), TrkB (for its acronym in English “tyrosine kinase-related B”) receptor gamma aminobutyric acid (GABA)), changes in neurotransmitters (dynorphin, enkephalins, GABA) (Nogushi et al, 1992).

5. A reorganization of neuronal structure resulted in the development of axon collaterals branching that increase the surface area or nociceptive afferent turn increasing glutamate release into the synaptic gap. (Terman and Bonica, 2001).

6. The loss of efficacy of inhibition produced by the descending pathways, with a decreased release of endogenous opioids, and even cellular degeneration of downstream neurons, indirectly also increases the frequency of the signal sent to the centers top (Doubell et al, 2003).

7. Glial activation, as both microglia are activated astrocytes observed: recruitment of microglia and astrocytes dorsal horn level, an increase in the cellular level expression and release of proinflammatory cytokines (interleukin-1 (IL1), factor tumor necrosis factor alpha (TNF α)), increase in the number of receptors on the cell surface (receptors for cytokines, NMDAR, APMPAR (for its acronym in English “α-amino-3-hydroxy-5-methyl-4-isoxazolepropionic acid receptor”), mGluR, among others), deregulation of glutamate uptake by the astrocyte (mediated by glutamate synthase 1 (Glu1) and glutamate—aspartate transporter (GLAST for its acronym in English, “glutamate aspartate transporter”)) and an increase in cell proliferation of microglia and astrocytes (Watkins et al, 2001).

Alterations in neural and molecular circuits are largely responsible why drugs that inhibit acute pain typically have reduced efficacy in the treatment of chronic pain (Paeile y Bilbeny, 2005).

Glial Role in Chronic Pain

In the last decade, numerous studies have shown that the classical notion that “pain is produced and maintained only by neurons” is a mistaken notion, because the glial cells in the spinal cord (astrocytes and microglia) would have a crucial role in the development and persistence of chronic pain (Meller et al, 1994; Milligan y Watkins L, 2009).

The association between glia and chronic pain is evident in the following experimental evidence:

1. Astrocytes and microglia dorsal horn of the spinal cord are activated against a variety of conditions that cause chronic pain and hyperalgesia, such as subcutaneous inflammation (Fu et al, 1999), subcutaneous administration of inactivated mycobacteria (Sweitzer et al, 1999), peripheral nerve trauma (DeLeo et al, 1999; Mains y Waxman; 2006), among others(Garrison et al, 1991).

2. Glial activation occurs by molecules from nociceptive afferent nerve endings (excitatory amino acids, SP, ATP), as well as second order neurons (nitric oxide, NO; prostaglandins; fractalkinas) (Carmignoto et al, 2000).

3. Activated glial cells release different neuroactive molecules capable of inducing or magnify pain, such as, NO prostaglandins, leukotrienes, arachidonic acid, excitatory amino acids (glutamate, aspartate, cysteine, quinolinic acid), growth factors, and enkephalinase and a variety of proinflammatory cytokines such as interleukin-1beta (IL-1β), interleukin-6 (IL-6) and tumor necrosis factor (TNF) (Kreutzberg G W, 1996; Watkins et al, 2002).

4. Glial cells and neurons have receptors for cytokines. Indeed, today it is accepted that cytokines have a role as a neuromodulator in the central nervous system. A level of nociceptive neurons of second order, IL-1β is able to increase the response by stimulation of C fibers, as well as central sensitization or Wind-up cord (Constandil et al, 2003), level nociceptive afferent endings, IL-1β release SP increases and probably glutamate (Inoue et al, 1999).

5. The algesic effects of proinflammatory cytokines on neurons and glia with specific antagonists or blocking antibodies (Sweitzer et al, 2001).

6. And in addition to classic neurotransmitters released by glial, already mentioned above, known as glial gliotransmitters as free D-serine (Ds) molecules. Ds is a dextrorotatory amino acid type (D) acting on the glutamate receptor (NMDA glycine site), suggesting a possible role of this gliotransmitter in the transmission of pain signals (Miller, 2004)

All the above background make glial cells an important modulator of nociception and therefore a potential target for drug therapies for chronic pain management. It is in this context that the strategy of suppressing the activity of glia at the spinal level (DeLeo et al, 1999) and thereby suppress the release of cytokines and restore the normal level of neuronal excitability appears.

In the literature it described various drugs capable of inhibiting the activity of glia with analgesic effects. The analgesic effect of inhibitors of glial cells are observed in different models of chronic neuropathic and inflammatory pain. These drugs can be found at: minocycline (Amin et al, 1996; Tikka et al, 2001), fluorocitrate (Hassel et al, 1992; Fonnum et al, 1997), ibudilast (AV411) (Ledeboer et al, 2007) and methylxanthines as PPT (3-methyl-1-(5-oxo-hexyl)-7-propylxanthine or propentofylline); (Si et al, 1998; Tawfik et al, 2008) and PTX (3,7-dimethyl-1-(5-oxo-hexyl)xanthine or pentoxifylline); (Liu et al, 2007; Mika et al, 2007; Mika, 2008).

These inhibitors methylxanthines as propentofylline (PPT) and pentoxifylline (PTX) are the most studied in relation to treatment of chronic pain due to readily cross the blood brain barrier and inhibit both the microglia and astrocytes.

Pentoxifylline (PTX) or fancy name, pentoxifylline (Aventis) is an FDA-approved drug and has been used for treating peripheral vascular disease, congestive cardiac and cerebrovascular insufficiency, sickle cell disease and diabetic neuropathy (Porter J et al, 1982; Frampton, 1995; Shen et al, 1991).

At the cellular level and PPT PTX block the reuptake of adenosine (Parkinson et al, 1991 y 1993; Tawfik et al, 2008) and inhibit the enzyme phosphodiesterase type I to IV as specified (Sweitzer et al, 2001).

Furthermore, propentofylline (PPT) has been used experimentally in testing for treating dementia in Alzheimer (Schubert P et al, 1998; Chauhan et al, 2005) and some cases of chronic pain (Sweitzer y DeLeo, 2011).

In vitro (Schubert et al, 2000) as well as in vivo (DeLeo et al, 1987) propentofylline mechanism (PPT) seems to be varied, as has also been shown to inhibit the production of free radicals (Rother Met al, 1996) and reduces the activation of glial cells and astrocytes by inhibiting glutamate release (Andine et al, 1990) and increased secretion of neurotrophins such as nerve growth factor (NGF, for its acronym in English “nerve growth factor”) (Shinoda et al, 1990).

Specifically in the field of pain, Schubert group reported that primary cultures of activated microglia by lipopolysaccharide (LPS) and treated with propentofylline, show inhibition of the production of tumor necrosis factor (TNF), IL-1β and radicals oxygen (Schubert y Rudolphi, 1998).

Furthermore, there has been a concomitant increase in AMPc and interleukin 10, a cytokine with anti-inflammatory properties. (Platzer et al, 1999; Detloff et al, 2008).

The biggest disadvantage of using inhibitors as analgesic glia is that for high levels of analgesia, these must be administered continuously for several days.

Data from our laboratory show that administration of PTX for 10 days lowers the threshold of pain, pressure test the leg of rats subjected to a model of monoarthritis, up to 21 days. In summary inhibition produced by glial pentoxifylline, if makes it ideal for administration in a system of sustained drug release over time.

Data shown together suggest that activation of glial cells in response to intense painful stimuli is responsible for the functional changes that occur in neurons and which result in maintaining the painful sensation in time (Paeile y Bilbeny, 2005). Therefore, drugs capable of inhibiting the activation of glia are important for the treatment of chronic pain and study new pharmaceutical forms that allow a continuous release over time, it becomes crucial to enhance the effectiveness of this drug type. One way to accomplish this is to load drugs that block the activity glial nanoparticles slowly disintegrate and permit the drug to be released for a period of time.

Polymeric Nanoparticles as Drug Delivery System

In recent years the growth and applications in the area of nanoscience and nanotechnology have been unprecedented. This increase has been observed in many levels but particularly in the field of medicine, offering significant advances in the diagnosis and treatment of diseases. As they have also been observed in new drug development, improving nutraceutical and production of biocompatible materials. (Duncan et al, 2003; DeJong et al, 2005; European science foundation (ESF) 2005; European Technology Plataform on Nanomedicine (ESP), 2005; Ferrari, 2005)

The reason why nanoparticles are attractive for use is the feature that has the relationship with the surface versus its mass, which is much higher compared with other molecules and ability to adsorb and transport other molecules.

The release of drug molecules from extended release as nanoparticles (NPs), made from biodegradable and biocompatible polymers is generating a strong impact on the preclinical development of new pharmaceutical forms (Born y Muller-Schulte, 2008). Such therapeutic molecules offer a wide platform to reduce the number of doses, reduce toxicity without altering its therapeutic effects, protect the drug from inactivation (due to protein binding or metabolism of the drug), and provide a sustained release stable for long periods of time and have greater specificity against target tissues (given by the functionalization of the molecule).

Among the prolonged-release systems using polymeric drugs NPs stands, which when arranged in a target tissue is maintained at the injection site to release the drug contained therein for long periods of time; and being the biggest advantage their biodegradability and biocompatibility, property is given mainly by the characteristic of the polymer used in its formulation. Different families of biodegradable polymers have been described in formulating NPs such as phospholipids, lipids, lactic acid, dextran, quitosina, etc. (Cascone et al, 2002; Baran et al, 2002; Duncan et al, 2003; Kipp et al, 2004).

The NPs exhibit good biocompatibility quitosina, however, its widespread use is limited by their antigenic and poor control of release of potential therapeutic agent, because its degradation is dependent on the enzyme activity (Duncan, 2003). Polyesters (lipids and phospholipids) polymers are widely used in the development of drug delivery systems, because they experience a suitable release profile, high biocompatibility and its degradation products are bio-absorbable. However, its use has been developed mainly to the area of cosmetics (ESP, 2005). The lactic acid polymer (poly (lactic acid) (PLA) and poly (lactic-glycolic acid) (PLGA)) and its copolymers biomaterials are excellent for encapsulating drugs in polymeric NPs (Waeckerle-Men et al, 2005; Cho H et al, 2004; Kumar et al, 2001; Ilium et al, 1987; Berchane et al 2007).

The Food and Drug Administration (FDA) approved the PLGA polymer as a biodegradable and biocompatible polymer. These polymers have been used for decades in practice for clinical applications in the area (Putney et al 1998), including prosthetic devices, implants, and drug-loaded microspheres for sustained release.

In the presence of water, PLGA is degraded to lactic and glycolic acid, which are natural byproducts molecules of various metabolic processes in the body. The biocompatibility of PLGA makes it an excellent candidate for the safe and non-irritating to load inside drug use. Moreover, pending PLGA controlled release characteristics, given by reason of its composition from lactic acid-glycolic acid.

The methodologies described in the literature for the development of systems NPs of biodegradable and biocompatible polymers are multiple, among which are: (i) Phase separation of the polymer (Thread et al 2004); (ii) Spray drying (Husmann et al 2002); (iii) Simple o doble emulsión—evaporación (Li X et al, 2009).

Furthermore, the release of an encapsulated drug in three phenomena may occur: (i) diffusion of the drug, (ii) degradation (iii) and erosion of the polymer matrix (Goepfirich A et al 1996).

Diffusion of the drug loaded into the PLGA NPs occurs as it generates the rupture of ester bonds at random. This phenomenon joined drug solubilization disposed on the surface of the NP or its porosity could generate an initial burst of release (burst release). Once it begins to decrease the mass of the particle, the sustained release phase in which the degradation and erosion of the polymer matrix occurs starts. Sustained release systems based drugs NPs possess unique properties depending on the preparation conditions and polymer properties and drug (Ito F et al, 2007; Freiberg et al, 2004; Li M et al, 2008; Ruan et al 2002).

The present invention involves the development and characterization of nanoparticles of biodegradable and biocompatible polymer (lactic acid-glycolic) acid and poly drug pentoxifylline to generate a new pharmaceutical form (nanoparticle-drug) allowing the reduction to a single dose for treatment effective chronic pain and preventing the onset thereof.

State of the Art

The document WO9702022 describes biocompatible and biodegradable for absorption of proteinaceous drugs and nanoparticles administration. The invention relates both to the method for producing said nanoparticles to use. Claimed nanoparticles for proteinaceous drugs or peptides with a particle size between 0.02 and 1 micron containing a biocompatible polymer selected from the group consisting of polyethylene, caprolactone, polylactide, polyglycolide, or a copolymer thereof. Preferably used are poly (D,L) lactic acid, poly(D,L) lactic co-glycolic particularly in a molar ratio of 50:50(%). in this document the particular use of pentoxifylline PLGA is not described.

The document WO2008054042 discloses the use of biodegradable PLGA nanoparticles to encapsulate ciprofloxacin, which has a sustained release property and the method of manufacturing the composite PLGA+ciprofloxacin. Biodegradable PLGA nanoparticles encapsulating ciprofloxacin have a particle size of 100-500 nm. Porous biodegradable nanoparticles claimed poly (DL-iactic-co-glycolic) encapsulating ciprofloxacin, and having a prolonged release property, and a particle size of 100-500 nm. Moreover it claims the manufacturing process and only the nanoparticle using ciprofloxacin with nanoparticles.

In this document the particular use of pentoxifylline PLGA is not described.

The document WO2007127363 discloses a method for depositing a coating comprising a polymer and at least two pharmaceutical agents to a substrate. Demands that the polymer to be used can be selected from the following: PLA, PLGA, PGA and Poly (dioxanone). Drugs that are claimed as rapamycin macrolide immunosuppressant and derivatives, in any case the polymer with pentoxifylline as a pharmaceutical agent described.

The document WO2009147372 discloses a non-water soluble composition for drug delivery comprising a conjugate and a matrix polymer. Where exposure of the composition to electromagnetic radiation at a predetermined wavelength and intensity, induce the release of active ingredient from the composition. The conjugate binds to the polymer matrix by non-covalent interactions. Vindicates not water-soluble composition in the form of a tablet, capsule, suspension, cream, ointment, lotion, powder, gel, solution, paste, spray, foam, oil, enema, suppository, controlled release matrix or depot or slow. The polymers constituting the composition may be selected from: poly(ethylene), poly(propylene), polyvinyl chloride, polyvinyl pyrrolidone, poly(2-hydroxyethyl methacrylate), poly(methyl methacrylate), poly(methacrylic acid), poly(acid acrylic), poly(diethylaminoethyl), poly(diethylaminoethyl), silicone, styrene-isoprene/butadiene-styrene, poly(lactic acid), poly(glycolic acid), poly(lactic acid-co-glycolic acid), poly (caprolactone), poly(orthoesters) and polyphosphazine. Drugs that can be used in the composition are antibiotics, analgesics, vitamins, antihistamines, anti-inflammatory. In this document the particular use of pentoxifylline as an active ingredient of the composition is disclosed.

The document WO2010009335 describes an implantable medical device, comprising: a substrate and a coating to it, the coating comprises at least one polymer and at least one pharmaceutical agent as substrate, in a therapeutically desirable morphology. Claims the medical device comprising the substrate and coating polymers. Also mentions ail the tools that can be used to insert the medical device. Claimed different drugs including pentoxifylline. The device comprises at least one of the following polymers, mixtures or derivatives thereof, polylactic (PLA); PLGA (poly(lactic-co-glycolic acid), polyanhydrides, polyorthoesters, poly(N-(2-hydroxypropyl)methacrylamide); DLPLA-poly(D,L-lactide); LPLA-poly(L-lactide); PGA-polyglycolic; PDO-poly(dioxanone); PGA-TMC-poly(glycolic-co-trimethylene carbonate); PGA-LPLA-poly(L-lactide-co-glycolide); PGA-DLPLA-poly (D,L-lactic-co-glycolic); LPLA-DLPLA-poly(lactic-co-D,L-lactic acid), and PDO-PGA-TMC-poly(glycolic-co-trimethylene carbonate-co-dioxanone) This paper describes the use not pentoxifylline especially as substrate or pharmaceutical agent.

WO2008128123 document includes compositions and methods for manufacturing a polymeric nanoparticle for targeted drug delivery, in the claims it is stated that the polymer may be poly-lactic acid, poly glycolic acid, poly-lactic-co-glycolic acid, and combinations thereof. Drugs that can be used in the invention, which relate to cancer drugs, antibiotics, antivirals, antifungals, anthelmintics, nutrients, siRNA, antibodies and antioxidants are also mentioned. However, no claims or particular drugs active ingredients mentioned.

The document WO2010111238 discloses a composition comprising a poly (alpha-hydroxy acid) substantially free of acid impurities, wherein the poly (alpha-hydroxy acid) is selected from poly(D,L-lactic-co-glycolic acid), poly(L-lactic acid), poly(D-lactic acid) and poly(D,L-lactic acid), in the document the level of impurities that may be mentioned composition, and the different proportions between the different groups described above. The use of this composition as a medical device such as stents (for example, vascular stents), electrodes, catheters, leads, implantable pacemaker, cardioverter or defibrillator housings, screws, rods, ophthalmic implants, femoral pins, bone plates, grafts claimed, anastomotic devices, perivascular wraps, sutures, staples, shunts for hydrocephalus, dialysis grafts, colostomy devices, fixing tubes ear drainage bag driver for pacemakers and implantable cardioverter and defibrillators, vertebral disks, bone nails, anchors suture, hemostatic barriers, clamps, screws, plates, clips, vascular implants, tissue adhesives and sealants, tissue scaffolds, various types of dressings (for example wound dressings), bone substitutes, intraluminal devices, and vascular supports. Highlighting variety of drugs and pentoxifylline pentifylline claimed, but for purposes other than the present invention. This paper presents the use of PLGA with pentoxifylline, but in no case a nanoparticle-drug presented as formulation for the treatment of chronic pain. Considering further that this document addresses a technical problem different from that proposed, this document does not affect the novelty or inventive step of this innovation. The document WO2005084710 describes nanoceldas allowing the sequential delivery of two different therapeutic agents. The nanocelda can be formulated in a pharmaceutical composition for delivery to patients suffering from diseases such as cancer, inflammatory diseases such as asthma, autoimmune diseases such as rheumatoid arthritis, infectious diseases, and neurological diseases such as epilepsy. The use of a polymer for nanoceldas PLGA as claimed. Nanocelda mentioned that can be administered by oral, parenteral, intravenous, inhalation, intramuscular, subcutaneous, rectal, intrathecal, nasal, vaginal, intradermal, mucosal or transdermally. This document pentoxifylline particular reference is not made.

The document The mechanisms of drug release in poly(lactic-co-glycolic acid)-based drug delivery systems—a review” (Fredenberg S, Wahlgren M, Reslow M, Axelsson A. Int J Pharm. 2011 Aug. 30; 415(1-2):34-52. Epub 2011 May 27), It is a review on the use of the polymer poly(D,L-lactic-co-glycolic acid) (PLGA), often used as a compound in biodegradable controlled release of encapsulated drugs. The release mechanism and the various encapsulation techniques are described. Physico-chemical influencing the rate of drug release processes are also described. A look is given to basic research, general and mechanistic application in the development of controlled-release pharmaceutical products.

The document “Functionalized poly(lactic-co-glycolic acid) enhances drug delivery and provides chemical moieties for surface engineering while preserving biocompatibility” (Bertram J P, Jay S M, Hynes S R, Robinson R, Criscione J M, Lavik E B. Acta Biomater. 2009 October; 5(8):2880-71. Epub 2009 May 4), describes the use of PLGA and modifications thereto, to enhance the release of various types of drugs In vivo.

The document “Biodegradable poly(lactic-co-glycolic acid) microparticles for injectable delivery of vaccine antigens” (Jiang W, Gupta R K, Deshpande M C, Schwendeman S P. Adv Drug Deliv Rev. 2005 Jan. 10; 57(3):391-410), describes using biodegradable PLGA as injectable polymeric particles, to control the release of antigens from a vaccine, in order to reduce the number of doses in the immunization program and optimize the desired immune response. He mentions that PLGA microparticles showed unprecedented flexibility and security, to carry out the release of one or multiple antigens from different physical-chemical and immunological characteristics requirements.

The document “PLGA/PEG-derivative polymeric matrix for drug delivery system applications: Characterization and cell viability studies” (Fernández-Carballido A, Pastoriza P, Barcia E, Montejo C, Negro S. Int J Pharm. 2008 Mar. 20; 352(1-2):50-7. Epub 2007 Oct. 16), studied the effect of the inclusion of an additive (PEG-derivative) in the final characteristics of the polymer matrix of PLGAs. Also cytotoxicity studies conducted in various cell cultures.

The document “PLGA-based nanoparticles: an overview of biomedical applications” (Danhier F, Ansorena E, Silva J M, Coco R, Le Breton A, Preat V. J Control Release. 2012 Jul. 20; 161(2):505-22. Epub 2012 Feb. 4). It has developed advances in the different supply systems based drugs PLGA nanoparticles for the treatment of different pathologies. Focus on nanoparticles that are suitable for parenteral use compared with microparticles are generally used as an implant. We describe and illustrate recent advances in the biomedical industry using nanoparticles.

The PLGA has been widely used for encapsulating drugs, however, no disclosure of a formulation for the treatment or prevention of chronic pain that corresponds to a PLGA nanoparticle containing pentoxifylline known.

### BRIEF DESCRIPTION OF THE INVENTION

The present invention discloses a new pharmaceutical formulation comprising polymeric nanoparticles of biodegradable polymer and biocompatible poly(lactic acid-glycolic acid) (PLGA) loaded with drug pentoxifylline and use of this new dosage form for effective treatment of chronic pain relief and prevention of chronic pain by the administration of a single dose.

In a preferred embodiment, the nanoparticles are synthesized by the method of double emulsion evaporation and modified comprising the steps of:

a) dissolving PLGA polymer in a solvent;

b) dissolving in purified water pentoxifylline;

c) addition of pentoxifylline dissolved in step b) the dissolved PLGA polymer in step a) previously cooled;

d) emulsifying the mixture of step c);

e) adding a solution of an emulsifying agent in purified water;

f) homogenizing the mixture of step e);

g) diluting the homogenization of step f) in purified water;

h) evaporation of the solvent in which the PLGA polymer is dissolved in step a);

i) washing the nanoparticles with purified water;

j) collection of nanoparticles in solution.

PLGA nanoparticles loaded with pentoxifylline of the present invention are useful to alleviate and prevent chronic pain via intrathecal administration of a single dose.

The pharmaceutical formulation of slow, controlled, sustained nanoparticles loaded PLGA pentoxifylline release of the present invention comprises the solution loaded PLGA nanoparticles obtained pentoxifylline the method described above and any additives, or pharmaceutically suitable auxiliary agent.

### BRIEF DESCRIPTION OF THE FIGURES

FIG. 1: (A) Size of the n-PLGA and n-PLGA-PTX determined by dynamic light scattering. (B) Frequency distribution of n-PLGA-PTX. (C) Morphological analysis by transmission electron microscopy (TEM) of the n-PLGA-PTX (n=6).

FIG. 2: Preventive effect of chronic neuropathic pain in rats treated with n-PLGA PTX three days prior to the generation of the sural injury, in the figure, the number 1 corresponds to the day of administration of the n-PLGA PTX, and number 2 at the time of the sural nerve cut was made in the neuropathy and neuropathy+n-PLGA PTX groups. Control (n=9), neuropathy (n=9) and neuropathy-PLGA PTX+n (n=5).

FIG. 3: Preventive effect of chronic pain in rats subjected to subcutaneous high frequency electrical stimulation (SHFS) and treated with n-PLGA PTX. In the figure, the number 1 corresponds to the day of administration of the n-PLGA PTX, and number 2 at the time the HFS was conducted in the SHFS and SHFS+n-PLGA PTX groups. Control (n=9), SHFS (n=12) and n-PLGA SHFS+PTX (n=5).

FIG. 4: Effect of treatment with revertive n PLGA-PTX in neuropathic rats, in the figure, the number 1 corresponds to the day of the sural nerve cut was made in the neuropathy and neuropathy+n-PLGA PTX groups; and number 2 is the day of administration of the n-PLGA PTX. Control (n=9), neuropathy (n=9) and neuropathy-PLGA PTX+n (n=6).

FIG. 5. Revertive Effect of treatment with PLGA-PTX n in rats subjected to HFS. In the figure, the number 1 is the day when HFS was conducted in groups LTP LTP awareness and PTX+n-PLGA; and number 2 is the day of administration of the n-PLGA PTX. Control (n=9), LTP sensitization (n=12) and LTP-PLGA PTX+n (n=3).

FIG. 6. Revertive Effect of treatment with PLGA-PTX n in rats with monoarthritis, in the figure, the number 1 is the day when the n-PLGA group were administered PTX (Monoarthritis+n-PLGA PTX). Monoarthritis or control (n=3), Monoarthritis+PTX (n=3) and Monoarthritis +n-PLGA PTX (n=3).

### DETAILED DESCRIPTION OF THE INVENTION

The present invention discloses a new pharmaceutical formulation comprising polymeric nanoparticles of biodegradable polymer and biocompatible poly (lactic acid-glycolic acid) (PLGA) loaded with drug pentoxifylline and using this new pharmaceutical form for the effective relief of chronic pain and for prevention of chronic pain by the administration of a single dose.

Nanoparticles PLGA polymers such polyesters are suitable as systems controlled and sustained release of drugs due to their release profile, its high biocompatibility since their degradation products are bioabsorbable.

The present invention utilizes encapsulation pentoxifylline, an analgesic that inhibits activation of the glial pain, microglia and astrocytes, inhibiting the release of cytokines in general and in particular interleukins. Drugs that inhibit glial activation, as the case of pentoxifylline, have shown to be effective in treating chronic pain, but this is dependent analgesic effect in repeated administration or chronic.

Using loaded PLGA nanoparticles pentoxifylline solves this problem by allowing the slow, controlled, sustained release of drug as the nanoparticles are degraded in the target tissue, so that administration of a single dose or alleviating effect has preventing chronic pain for several days.

Nanoparticles loaded PLGA pentoxifylline of the present invention are synthesized by any method known in the art, for example, but not limited to: method of phase separation of polymer, method of spray drying, method of self-assembly (Chan, J M et al., 2009), or method and single or double emulsion evaporation, traditional or modified. In a preferred embodiment, the nanoparticles are synthesized by the method of double emulsion evaporation and modified comprising the steps of:

a) dissolving PLGA polymer in a solvent;

b) dissolving in purified water pentoxifylline;

c) addition of pentoxifylline dissolved in step b) the dissolved PLGA polymer in step a) previously cooled;

d) emulsifying the mixture of step c);

e) adding a solution of an emulsifying agent in purified water;

f) homogenizing the mixture of step e);

g) diluting the homogenization of step f) in purified water;

h) evaporation of the solvent in which the PLGA polymer is dissolved in step a);

i) washing the nanoparticles with purified water;

j) collection of nanoparticles in solution.

The PLGA polymer used in step a) and therefore PLGA nanoparticles of the present invention, have a range of ratio of polylactic acid to polyglycolic acid from 10% to 90% polylactic acid polyglycolic acid, up to 90% of 10% polylactic acid with polyglycolic acid may be any combination that falls within these ranges. In a preferred embodiment, the ratio is 50% polylactic acid and 50% polyglycolic acid.

The solvent used in step a) is any which allows dissolving the polymer, for example, but not limited to dichloromethane or chloroform.

The polymer of PLGA to pentoxifylline used in the synthesis method of the present invention ranges from 0.01% and 0.0003% pentoxifylline PLGA, PLGA to 9% and 0.3% pentoxifylline, which may be any combination that is around this range, in a preferred embodiment, the ratio of polymer to pentoxifylline PLGA used in the synthesis method of the present invention is 0.3% and 0.01% PLGA pentoxifylline.

Step d) emulsifying the mixture of step c) is carried out with any method known in the art. (for example. Cheng, L. et al 2011) In a particular embodiment step d) emulsifying the mixture of step c) is performed using a sonicator. In a preferred embodiment, the emulsion in the sonicator is performed at a frequency of 10 to 30 kHz, at a power of 90-170 watts for 40 to 80 seconds. In another even more preferred embodiment, the emulsion in the sonicator is performed at a frequency of 20 kHz, at a power of 130 watts for 60 seconds.

The emulsifying agent used in the solution of step e) may be any that allows to emulsify the emulsion of step d), for example, but not limited to: polyvinyl alcohol (PVA), polyethylene glycol or derivatives thereof, or emulsifying cationic and anionic for pharmaceutical use, or any combination thereof.

In a particular embodiment, when PVA is used in step e), it is hydrolyzed in 80 to 95%. in a preferred embodiment, when PVA is used in step e), it is 87-89% hydrolyzed.

In another preferred embodiment, when PVA is used in step e), the PVA solution in purified water comprises 0.1 to 5% w/v PVA. in a further preferred embodiment, the PVA solution in purified water from step e) comprises 0.5% w/v PVA.

Homogenization of step f) is performed by any method known in the art (for example. Ribeiro-Costa, R M et al, 2004), for example, but not limited to homogenization using a sonicator, by vortexing, by an ultra-turrax, through high pressure homogenizers or by homogenizers of any kind or any combination thereof, in a particular embodiment, the homogenization of step f) is performed using a sonicator. In a preferred embodiment, the homogenization in the sonicator is performed at a frequency of 10 to 30 kHz, at a power of 90-170 watts, for 5 to 70 seconds, in another further preferred embodiment, the homogenization in the sonicator is performed at a frequency of 20 kHz, at a power of 130 wafts for 15 seconds and then repeat for 40 seconds

Evaporation of the solvent in step h) is performed by any method known in the art (for example: Chen, J, & Davis, S S 2002), for example but not limited to: stirring the solution, and nitrogen gas streams or oxygen, heat, freeze drying or any combination thereof. In a particular embodiment, the evaporation of the solvent in step h) is effected by moderate orbital shaking (70 to 170 rpm) at room temperature for 5 to 20 hours.

The collection of the nanoparticles in solution from step j) is performed by any method known in the art (for example. Shi, J., et al 2011), for example but not limited to: filtration, centrifugation and filtration, centrifugation differential gradient centrifugation or any combination thereof. In a particular embodiment the nanoparticles are collected by filtration and centrifugation. In a preferred embodiment, the filter for collection of nanoparticles is performed by a filter with 80 to 120 KDa cut and centrifuging at 3500 to 5500G for the time necessary to obtain a solution with nanoparticles. In a further preferred embodiment, the filtration for collection of nanoparticles is performed by a 100 kDa cut filter and centrifuging at 4500G for the time necessary to obtain a solution with nanoparticles.

PLGA nanoparticles loaded with pentoxifylline of the present invention have a homogeneous size from 150 to 410 nm. In a preferred embodiment, the nanoparticles loaded PLGA pentoxifylline of the present invention have a homogeneous size of 250 nm.

PLGA nanoparticles loaded with pentoxifylline of the present invention are spherical, with a smooth and hydrophilic surface, a polymeric shell and a central cavity that is encapsulated pentoxifylline.

PLGA nanoparticles loaded with pentoxifylline of the present invention have a negative surface charge with a value between −24 and −13 mV. In a preferred embodiment, the nanoparticles PTX loaded PLGA of the present invention possess a negative surface charge of −18.5 mV value.

The synthesis method of nanoparticles loaded PLGA pentoxifylline of the present invention enables encapsulation efficiency of between 40 and 60% of the drug added. In a preferred embodiment, the encapsulation efficiency is 50% of the drug added.

The proportion of pentoxifylline encapsulated in PLGA nanoparticles of the present invention is between 1 .mu.g in 10 .mu.l of pentoxifylline nanoparticle solution until 3 μg of pentoxifylline in 10 .mu.l of solution of nanoparticles or any ratio that falls within these ranges, in a preferred embodiment the proportion of pentoxifylline encapsulated in PLGA nanoparticles of the present invention is pentoxifylline 2 μg in 10 .mu.l of solution of nanoparticles.

PLGA nanoparticles loaded with pentoxifylline of the present invention are stable exhibiting no significant change in size when exposed to low temperature or lyophilization.

PLGA nanoparticles loaded with pentoxifylline of the present invention are useful for treating and preventing chronic pain by administering a single dose.

PLGA nanoparticles loaded with pentoxifylline of the present invention are administered in a single dose. The single dose of nanoparticles loaded PLGA pentoxifylline of the present invention is administered for example, but not limited to: intrathecally, intravenously or intramuscularly. In a preferred embodiment, the single dose of nanoparticles loaded PLGA pentoxifylline of the present invention is administered intrathecally.

The pharmaceutical formulation of slow, controlled, sustained nanoparticles loaded PLGA pentoxifylline release of the present invention comprises the solution loaded PLGA nanoparticles obtained pentoxifylline the method described above and any additives, or pharmaceutically suitable auxiliary agent.

The pharmaceutical formulation single dose of slow, controlled, sustained PLGA nanoparticles loaded with pentoxifylline pharmacological effect release of the present invention it is in the range of 0.001 to 0.1 mg of pentoxifylline encapsulated/kilo of body weight of the subject suffering or known to suffer chronic pain. In a preferred embodiment, single-dose, controlled and sustained PLGA nanoparticles loaded with pentoxifylline pharmacological effect of the present invention slow release pharmaceutical formulation is encapsulated pentoxifylline 0.01 mg/kilo of body weight of the subject suffering or known to suffer chronic pain.

### Advantages of the Invention

The present invention solves the problem of the need for continuous administration of drugs to treat chronic pain. The present invention utilizes a single dose of a pharmaceutical formulation comprising at least a solution of biodegradable and biocompatible polymeric nanoparticles loaded PLGA pentoxifylline for preventing and treating chronic pain and can further comprise any additive or pharmaceutically suitable auxiliary agent.

### INDUSTRIAL APPLICATION

The present invention applies to medicine industry to cover all those diseases that result from the pathology of chronic pain generate background.

### EXAMPLES

### Example 1

### Synthesis and Characterization of Polymeric Nanoparticles Loaded with pentoxifylline (nPLGA-PTX)

Nanoparticles (NPs) loaded PLGA pentoxifylline (PTX-nPLGA) were synthesized by a modification of the method of double emulsion (water/oil/water phase) described by the group of Li (Li et al, 2001). The type of PLGA (Sigma) used comprises proportions of 50% polylactic acid and 50% polyglycolic acid. PLGA copolymer (3 mg/mL) dissolved in dichloromethane (Sigma), and the drug (PTX, Sigma) was dissolved in milliQ water. The dissolved drug (0.5 mL) was added to the PLGA solution (3 mg/mL) previously cooled with ice. Then, this mixture was emulsified with a sonicator (VCX 130, Sonic Vibracell, USA) at 100% amplitude (frequency 20 kHz, power 130 watts) for 80 seconds. To this emulsion was added a solution of 0.5% w/v polyvinyl alcohol (PVA, 87-89% hydrolysed, Sigma) dissolved in milliQ water and was homogenized twice at 100% amplitude (20 KHz, 130 watt ) 15 seconds and 40 seconds each time. This double emulsion to a beaker with milliQ water (25 mL) and dichloromethane present in the sample was eliminated by evaporation in a system moderate agitation (120 rpm on orbital shaker) overnight at room temperature was added. Free nanoparticles of dichloromethane, washed in milliQ water and collected by filtration using a cellulose filter with a 100 kDa cut (Amicon, Miilipore) by centrifuging at 4500 G, to obtain a solution with nanoparticles. Finally the pellet was stored at −20° C. To control the preparation process and the effects of the nanoparticle, empty nanoparticles were prepared, employing the same methods described above.

The size and zeta potential of the nanoparticles was measured by (dynamic light scattering) DLS (zetaziser nano S90, Malvern, Reino Unido) y su morfologfa por microscopía de transmision electrónica (TEM) (EVO MA10, Zeiss, Alemania). For this, an aliquot of 50 mL of a preparation of nanoparticles was taken and brought to a volume of 1 mL with milliQ water for analysis by DLS.

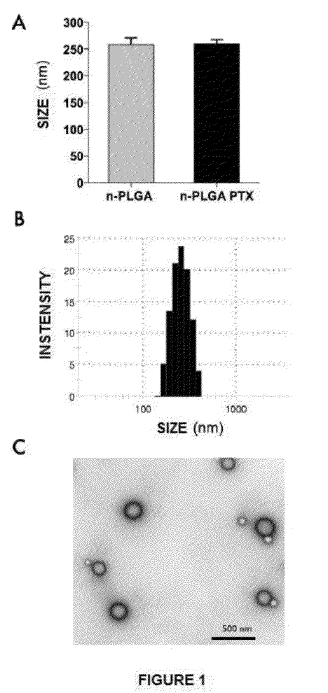

The n-PLGA PTX showed an approximate size of 256.8±79.5 nm (n=6, samples or separate preparations), which was measured by dynamic light scattering and showed no significant differences from the empty nanoparticles (n -PLGA), as shown in FIG. 1A. 1B shows the frequency distribution of PLGA-PTX n. The shape of the n-PLGA PTX was assessed by transmission electron microscopy (TEM) using as dye uranyl acetate, which allowed the hydrophilic areas highlight preparation. The images obtained showed spherical particles with a hydrophilic surface, a polymeric shell and a central cavity where the encapsulated drug according to their physicochemical properties and the literature, as shown in FIG. 1C. The analysis of the surface charge or zeta potential of the n-PLGA PTX by dynamic light scattering showed that the particles possess a negative charge with a value of −18.66±4.91 mV (n=5, independent preparations).

### Example 2

### Encapsulation Efficiency and In Vitro Release Kinetics

The encapsulation efficiency (EE %) of PTX was determined by quantitative comparison of the amount of drug between the initial charge and the rest of the drug remaining in solution after centrifugation of nPLGA-PTX, during the period preparation of nanoparticles. The amount of drug was determined by UV-visible spectrophotometry (Agilent 8453, Agilent thecnologies, Alemania) a una longitud de onda de 273 nm en cubeta de cuarzo semimicro o UPLC (Acquity system, Waters, Milford, Mass., USA).

Chromatographic separation of the PTX was performed on a C18 column of dimensions 50×2.1 mm, 1.7 μm; mobile phase had a ratio 89:1:10 (v/v) water, methanol and acetonitrile at a flow rate of 0.6 mL/min. Detection was obtained at a wavelength of 273 nm, with an injection volume of 5 uL. EE % was determined from the following relationships:

Theoretical load capacity=total drug/(total drug+polymer) (i)

Actual load capacity=encapsulated drug/(total drug+polymer) (ii)

EE %=(actual load capacity/theoretical load capacity)×100% (iii)

The encapsulation efficiency obtained showed that 50% of drug added in the formulation was encapsulated. From these results, the real or effective charge (English loading) was determined and calculated doses to be used in the in vivo experiments, having been established that 10 L of nanoparticles containing an amount of 2 μg of pentoxifylline (n=8 preparations independent).

### Example 3

### Determination of the Analgesic Properties and In Vivo Comparison of Nanoparticles

Animals

A total of 89 male Sprague-Dawley rats weighing 200-250 grams were used. All rats were obtained from the animal house of the University of Chile School of Medicine. The rats were kept in controlled conditions of light darkness (12:12, light: dark), temperature (22+3° C.) and airflow. Counted animals with food and water ad libitum. Daily cages were neat and animals were evaluated on their general condition (general appearance, weight and size). Ail experimental protocols were conducted according to the “Guide Care of Laboratory Animals” published by National Institutes of Health (NIH), the “guide for the ethical use of animals in research of pain” (Jayo Cisneros, 1996) published by International Association for Study of Pain (IASP) and the Ethics Committee of the University of Santiago, Chile.

Chronic Pain Models

Mononeuropathy

Experimental animals were anesthetized with isoflurane and through a small incision in the skin, approximately 1 cm long, thigh sciatic nerve was exposed. The subcutaneous tissue and muscle was debrided to find the sciatic nerve. Later the tour continued until the division of the nerve into three branches: the sural, common perineal and tibial. The branches were then separated and the sural nerve was ligated and divided to two millimeters of his birth. Finally a suture by planes was performed. Once completed the intervention were administered 5 mg/kg ip ketoprofen as analgesic, and 7.5 mg/kg, ip enrofloxacin as an antimicrobial (Rivera and Cabrero, 2008) once daily for three days. This generates a nerve injury hyperalgesia or chronic pain that can be compared to the pain that occurs in people suffering from lumbago and remains in animals for over a month. The animals were used for 14 days post surgery.

Subcutaneous High-Frequency Electrical Stimulation (SHFS, for its Acronym in English, Subcutaneous High Frequency Stimulation)

Subcutaneous model high-frequency electrical stimulation, SHFS, subcutaneous is high frequency electrical stimulation (HFS), which generates a peripheral pain without injury, which represents an ideal place to study for example pain associated with neuralgia model trigeminal. For this, rats were anesthetized with isoflurane 2.0% 1,5-, and inserted needle electrodes in the second and third toe of the right hind leg. Electrical stimulation was performed in two periods of three minutes separated by an interval of 10 minutes without stimulation, in each period, the rat was stimulated with trains of stimulation 1 second idle for 9 seconds. Electrical stimulation consisted of pulses at a frequency of 100 Hz and intensity of 120 volts 7 mA.

Rats were finally evaluated by behavioral Randall-Sellito test for 0, 1,3,7, 10, 14 and 21 days.

Monoartritis

Sprague-Dawley rats weighing approximately 100 g were used. This model was described by Butler (Butler et al, 1992). He was inoculated into the right paw (tibio-tarsal fourth) of rats with 50 uL of complete Freund's adjuvant containing 300 ug Mycobacterium butiricum. This injection produces a localized arthritis syndrome and found to be stable between four and six post-inoculation, weeks During this period there is an establishment of neurogenic pain and hyperalgesia, and this pain is held for more than two months period. Control rats were injected with the vehicle only Mycobacterium butiricum.

Intrathecal Injection

All doses of nanoparticles or free drugs were administered by intrathecal (it) in a maximum volume of 10 L, dissolved in artificial cerebrospinal fluid (LCA: 1.3 mm CaCl2, KCl 2.8 mm, 0.9 mm MgCl2, NaHCO3 20 mM, 2.5 mM Na 2 HPO 7H2O, 125 mM NaCl). The injection i.t. it involves administration of the drug into the subarachnoid space between lumbar vertebrae L5 and L6 (Mestre et al, 1994) using a Hamilton syringe with a needle 26Gx1/2”. The entrance to the subarachnoid space evidenced by a slight movement in the tail of the rat, due to the mechanical stimulation of the needle to penetrate the spinal cord meninges.

To proceed with this injection i.t. Rats were anesthetized briefly with isofluorane (5% in oxygen gas) for 2 minutes. For controls, an injection of 10 ul of LCA was conducted.

### Algesymetry

Evaluation of the analgesic effect of PTX-loaded nanoparticles or free drug was performed with the pressure test in rats paw using an algesimeter or equipment called Randall-Selitto (1957); (Ugo Basile, Italy). This test involves the gradual and increasing compression of the leg using a blunt tip. The nociceptive response is evidenced by the reflection of paw withdrawal or vocalization rat.

This test determines the nociceptive threshold (in grams) in response to mechanical stimulation. The studies were performed before (preventive) and/or after (curative) induction to a particular model of chronic pain (mononeuropathy, monoarthritis, SHFS).

Preventive Effect of n-PLGA PTX in In Vivo Models of Cronic Pain

The preventive effect of the n-PLGA PTX in the establishment of chronic pain and neuropathy model in the subcutaneous model high-frequency electrical stimulation (SHFS) was evaluated.

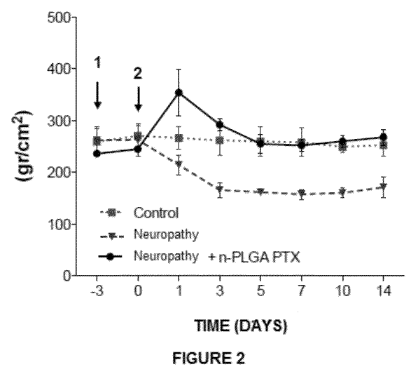

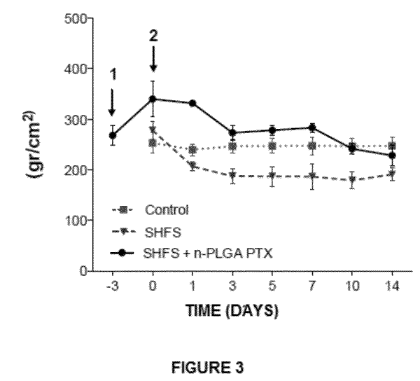

In both models, the experimental design was similar and consisted of the administration of the n-PLGA PTX three days before (FIGS. 2 and 3, marked with number 1, -3 days time) Generation pain model (FIGS. 2 and 3, marked with number 2, time 0 days). The evaluation of the animals was performed with the Randall-Sellito test, in the 2 models of chronic pain animals they were separated into three experimental groups: the control group (or sham) that corresponds to the animals underwent the same experimental other groups but without making induction painful picture surgical procedure. The positive control group, which corresponds to the animals that the painful picture was induced; ie in the case of the sural nerve neuropathy is cut, while in the case of the leg SHFS is electrically stimulated. The third group corresponds to the experimental group in which n PLGA-PTX is administered and then the model of chronic pain is generated, in ail cases the animals are evaluated through day 14 post induction of pain box.

The results obtained in both experimental models showed that the control group (n=9) showed no changes in pain threshold was expected. Moreover, rats subjected to neuropathy models (n=9) or SHFS (n=12) showed a significant decrease of the threshold. Interestingly, those groups previously treated with n-PLGA PTX and subsequently received a cut of the sural nerve (n=5) or the generation of SHFS (n=5) showed an increase in pain threshold that remains to this day 14 of the study, as shown in FIGS. 2 and 3, the results obtained in both experimental models support that treatment with PLGA-PTX n prevent chronic pain or SHFS induced neuropathy.

Therapeutic Effect (Revertive) of the n-PTX PLGA In Vivo Models.

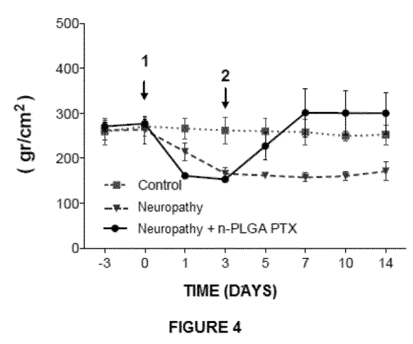

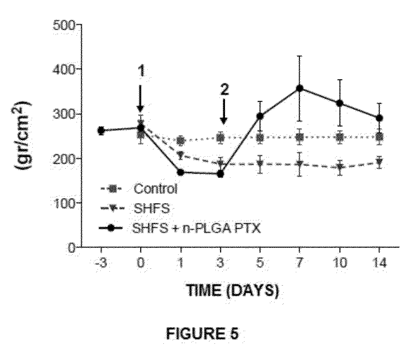

Using the same chronic pain models described above are developed an experimental design to test the therapeutic effect of PLGA-PTX n. For this, the n-PLGA administered 3 days after PTX (FIGS. 4 and 5, marked with number 2, time 3 days) to make a cut in the sural nerve stimulation or SHFS (FIGS. 4 and 5, marked with number 1 in time 0 days). The results showed that in both cases, the control group showed no changes in their pain threshold. Regarding the neuropathic model, rats that received a cut in the sural nerve (neuropathy group, n=9) from the second day showed a significant decrease of your pain threshold. Moreover, the rats in the group receiving treatment with n-PLGA PTX (n=5) after 3 days of receiving the court sural nerve showed a significant change in pain threshold, completely reversing neuropathy two days after administering the nanoparticles and which continues to the end of the experiment day 14. The results showed that treatment with PLGA-PTX n has a revertive mononeuropathy effect generated experimentally in rats as shown in FIG. 4.

The above results were corroborated with the model of high frequency stimulation. In FIG. 5, the results, which clearly shows that the administration of PLGA-PTX n reversed the decrease in pain threshold shown in rats subjected to SHFS shown.

The therapeutic effect of n-PLGA-PTX was also shown in a third model of chronic pain of monoarthritis obtained as indicated above. Butiricum Mycobacterium injection produces a localized arthritis syndrome and found to be stable for four to six weeks after inoculation, and neurogenic hiperalgésico and persistent pain states, this fable can be maintained for a period of two months. The pain is very stable and suitable for studying the effects of drugs with antinociceptive properties.

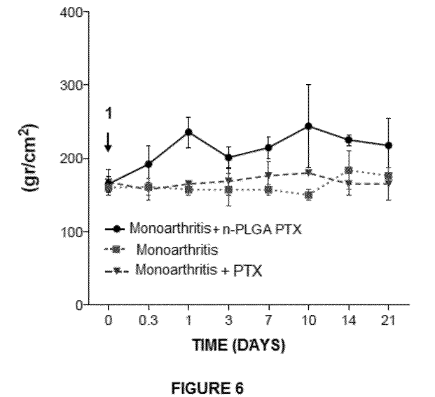

The experimental design was induced to the three experimental groups to the generation of monoarthritis. Groups of animals, one group received a single dose of pentoxifylline (Monoarthritis+PTX), another was controlling monoartritis and third (Monoarthritis+n-PLGA PTX) was treated with nanoparticles on day 0 (FIG. 6, marked with number 1, time 0 days).

The results (FIG. 6) show a clear increase of nociceptive threshold of the animals treated with nanoparticles, confirming the results obtained with the other two study models.

### Example 4

### Toxicological Tests

To study the possible toxic effects of nanoparticles assessment of biochemical and hematological blood parameters was performed. Blood samples from rats treated with nPLGA-PTX and their respective controls were obtained. In the biochemical profile parameters for the renal and hepatic metabolism together with an assessment of haematological parameters were analyzed.

Table 1 shows the results obtained, which shows no significant differences in the parameters analyzed at 3, 7 and 10 days after administration of nPLGA-PTX occur.

Similarly the measured hematological parameters showed no significant changes, as shown in Table 2.

TABLE 1
Toxicological analysis of blood biochemical parameters determined
at 3, 7 and 10 days after administration of n-PLGA-PTX.

| | | n-PLGA PTX | | |
|---|---|---|---|---|
| | Control | Day 3 | Day 7 | Day 10 |
| Parameter | (n = 12) | (n = 3) | (n = 3) | (n = 3) |
| Total Protein (gr/dL) | 5.9 ± 2.2 | 5.5 ± 0.9 | 6.2 ± 0.4 | 6.2 ± 0.1 |
| Albumin (gr/dL) | 3.3 ± 0.1 | 3.9 ± 0.4 | 3.5 ± 0.1 | 3.6 ± 0.1 |
| Globulin (gr/dL) | 3.4 ± 0.4 | 2.1 ± 0.7 | 2.7 ± 0.5 | 2.6 ± 0.1 |
| Phosphorus (mg/dL) | 8.7 ± 0.3 | 8.3 ± 0.4 | 7.9 ± 0.1 | 7.8 ± 0.0 |
| Calcium (mg/dL) | 9.9 ± 0.5 | 9.9 ± 0.2 | 10.2 ± 0.1 | 10.3 ± 0.1 |
| Bilirubin (mg/dL) | 0.08 ± 0.03 | 0.06 ± 0.01 | 0.07 ± 0.01 | 0.06 ± 0.01 |
| Alkaline phosphatase (U/L) | 268 ± 32 | 178 ± 1 | 184 ± 71 | 151 ± 32 |
| Lactate Dehydrogenase LDH (U/L) | 421 ± 81 | 494 ± 244 | 579 ± 59 | 392 ± 7 |
| GTT (U/L) | 3.4 ± 1.6 | 5.0 ± 2.8 | 4.5 ± 0.7 | 5.0 ± 0.0 |
| ALT/GTP (U/L) | 62.5 ± 4.5 | 73.0 ± 1.4 | 75.6 ± 2.7 | 72.6 ± 2.9 |
| AST/GOT (U/L) | 110 ± 4 | 113.5 ± 18.5 | 130.5 ± 18.8 | 103.4 ± 5.1 |
| Urea Nitrogen (mg/mL) | 16.5 ± 2.5 | 12.3 ± 1.3 | 12.5 ± 0.0 | 12.8 ± 0.9 |
| Creatinine (mg/mL) | 0.6 ± 0.006 | 0.30 ± 0.01 | 0.30 ± 0.00 | 0.35 ± 0.07 |

TABLE 2
Toxicological analysis of certain at 3, 7 and 10 days after
administration of PLGA-PTX n hematological blood parameters.

| | | n-PLGA PTX | | |
|---|---|---|---|---|
| | Control | Day 3 | Day 7 | Day 10 |
| Parameter | (n = 12) | (n = 3) | (n = 3) | (n = 3) |
| Total Leukocytes | 6.4 ± 1.8 | 5.7 ± 0.9 | 5.9 ± 0.9 | 5.7 ± 0.6 |
| (miles/μL) | | | | |
| Segmented(miles/μL) | 2.1 ± 0.6 | 1.4 ± 0.4 | 0.9 ± 0.2 | 1.3 ± 0.1 |
| Lymphocytes | 4.7 ± 1.5 | 4.2 ± 0.5 | 4.9 ± 0.7 | 4.1 ± 0.7 |
| (miles/μL) | | | | |
| Erythrocytes | 5.9 ± 1.0 | 7.1 ± 0.9 | 7.0 ± 0.1 | 7.2 ± 0.1 |
| (miles/μL) | | | | |
| Hematocrit (%) | 30.5 ± 7.5 | 40.8 ± 4.4 | 39.9 ± 1.2 | 40.9 ± 0.8 |
| Hemoglobin (gr/dL) | 12.4 ± 1.6 | 13.9 ± 1.3 | 13.5 ± 0.3 | 13.8 ± 0.3 |
| VCM (fL) | 59.9 ± 3.5 | 57.9 ± 1.2 | 57.1 ± 1.5 | 56.9 ± 0.5 |
| CHCM (gr/dL) | 27 ± 3.5 | 34.3 ± 0.4 | 33.8 ± 0.3 | 33.8 ± 0.1 |

### REFERENCES

Amin A, Attur M, Thakker G, Patel P, Vyas P, Patel R. Patel I, Abramson S. A novel mechanism of action of tetracycline: effects on nitric oxide synthases. Proc Natl Acad Sci USA. (1996), 93: 14014-14019.
Andine P, Rudolphi K A. Fredholm B B, Hagberg H. Effect of propentofylline (HWA 285) on extracellular purines and excitatory amino acids in CA1 of rat hippocampus during transient ischaemia. Br J Pharmacol (1990), 100: 814-818.
Baran E T, Özer N, Hasirci V. In vitro half-life of nanoencapsulated L-asparginase. J Mat in Med (2002), 13: 1113-1121.
Berchane N S, Carson K H, Rice-Ficht A C, Andrews M J. Effect of mean diameter and polydispercity of PLG microspheres and drug release: experiment and theory. Int J Pharm (2007).
Bertram J P, Jay S M, Hynes S R, Robinson R, Criscione J M, Lavik E B. Functionalized poly (lactic-co-glycolic acid) enhances drug delivery and provides chemical moieties for surface engineering while preserving biocompatibility. Acta Biomater. 2009 October; 5(8):2860-71. Epub 2009 May 4.
Born P J, Muller-Schulte D. Nanoparticle in drug delivery and environmental exposure: same size, same risk?, Nanomedicine (2006), 1:235-249.
Butler S. H., Godefroy F., Besson J.-M, Weil-Fugazza J. A limited arthritic model studies in a model of monoarthritis in the rat. Pain (1992), 48: 73-81.
Carmignoto G. Astrocyte neurone crosstalk variants of the same language. Trend Pharmacol Sci (2000), 10: 373-375.
Carmignoto G. Reciprocal communication system between astrocytes and neurones. Prog Neurobiol (2000), 6:581-581.
Cascone M G, Lazzeri L; Carmignani C, Gelatin nanoparticles produces by a simple W/O emulsion as delivery system for methotrexate. J Mat Sci: Mat in Med (2002), 13523-13526.
Chan, J. M, et al. PLGA-lecithin-PEG core-shell nanoparticles for controlled drug delivery. Biomaterials 30, 1627-1634 (2009).
Chauhan N B, Siegel G J, Feinstein D L. Propentofylline attenuates tau hyperphosphorylation in Alzheimer's Swedish mutant model Tg 2576. Neuropharmacol (2005), 48(1): 93-104.
Chen, J. & Davis, S. S. The release of diazepam from poly(hydroxybutyrate-hydroxyvalerate) microspheres. Journal of Microencapsulation 19, 191-201 (2002).
Cheng, L, Jin, C, Lv, W., Ding, Q. & Han, X. Developing a Highly Stable PLGA-mPEG Nanoparticle Loaded with Cisplatin for Chemotherapy of Ovarian Cancer, PLoS ONE 6, e25433 (2011).
Chizh B A. Novel approaches to targeting glutamate receptors for the treatment of the chronic pain. Aminoacid (2002), 23: 169-176.
Cho H, Chung D, Jeongho A. Poly(D-Lactide-ran-epsilon caprolactone)-poly(ethyleneglycol)-poly(D-L lactide-ran-epsilon caprolactone) as parental drug-delivery system. Biomaterials (2004), 25(17): 3733-3742.
Chou R, Huffman L H; American Pain Society; American College of Physicians. Medications for acute and chronic low back pain: a review of the evidence for an American Pain Society/American College of Physicians clinical practice guideline. Ann Intern Med. (2007), 147(7):505-14.
Codere T, Katz J, Vaccarino A, Melzack R. Contribution of central neuroplasticity to pathological pain: review of clinical and experimental evidence. Pain (1993), 52: 259-285.
Constandil L, Pelissier T, Soto-Moyano R, Mondaca M, Sáez H, Laurido C, Muñoz C, López Nandy, and Hernández A. Interleukin-1β increases spinal cord wind-up activity in normal but not in monoarthritic rats. Neuroscience Letters (2003), 342: 139-142.
Coutaux A, Adam F, Wilier J-C, Le Bars D. Hyperalgesia and allodinia: peripheral mechanisms. Joint Bone Spine (2005), 72: 359-371.
Danhier F, Ansorena E, Silva J M, Coco R, Le Breton A, Preat V. PLGA-based nanoparticles: an overview of biomedical applications. J Control Release. 2012 Jul. 20; 161(2):505-22. Epub 2012 Feb. 4.
DeJong W H, Geertsma R E, Roszek B. Nanotechnology in medical applications possible risk for human health. Report 265001 002/2005 National institute for public health and environment (RIVM). Bilthoven, The Netherlands.
DeLeo J, Toth L, Schubert P, Rudolphi K, Kreutzberg. G W. Ischemia-induced neuronal cell death, calcium accumulation, and glial response in the hippocampus of the Mongolian gerbil and protection by propentofylline (HWA 285). J Cereb Blood Flow Metab (1987), 7: 745-751.
DeLeo J A, Colburn R W. Proinflammatory cytokines in glial cells: Their role in neuropathic pain, in: Cytoquines and Pain (Progress in inflammation Research). Birkhauser Basel Switzerland (1999): 159-182.
Detloff M R, Fisher L C, McGaughy V, Longbrake E, Popovich P G, Basso D M. Remote activation of microglia and pro-inflammatory cytokines predict the onset and severity of below-level neuropathic pain after spinal cord injury in rats. Exp Neurol (2008), 212: 337-347.
Doubell T P, Mannion R J, Woolf C J. The dorsal Horn: state-dependent sensory processing, plasticity and the generation of pain. En: Wall P, Melzack R, editores. Textbook of Pain. 4th ed. Philadelphia: Churcill Livingstone (2003): 165-182.
Duncan R. The Dawning era of polymer therapeutics. Nat Rev Drug Disc (2003), 2:347-360.
ESF. European Science Foundation (2005) Policy briefing (ESF). ESF scientific forward look on nanomedicine (IREG). Strasbourg, France (ISBN 2-912049-520).
ESP. European Thecnology Plataform on Nanomedicine (2005). Vision paper and basis for a strategic research agenda for nanomedicine. European commission Luxemburg office for official publications of the European commission (ISBN 92-894-9599-5).
Fernández-Carballido A, Pastoriza P, Barcia E, Montejo C, Negro S. PLGA/PEG-derivative polymeric matrix for drug delivery system applications: Characterization and cell viability studies. Int J Pharm. 2008 Mar. 20; 352(1-2):50-7. Epub 2007 Oct. 18.
Ferrari M. Cancer nanothecnology: opportunities and challenges. Nat Rev Cancer (2005), 5: 161-171.
Fonnum F, Johnsen A,. Hassei B. Use of fluorocitrate and fluoroacetatein the study of brain metabolism. Glia (1997), 21: 106-113.
Frampton J E, Brogden R N, Pentoxifylline (oxpentifylline). A review of its therapeutic efficacy in the management of peripheral vascular and cerebrovascular disorders. Drugs Aging. 1995 December; 7 (6):480-503.
Freiberg S, Zhu X X. Polymer microspheres for controlled drug release. Int J Pharm. (2004), 282(1-2):1-18.
Fredenberg S, Wahlgren M, Reslow M, Axelsson A. The mechanisms of drug release in poly(lactic-co-glycolic acid)-based drug delivery systems-a review, int J Pharm. 2011 Aug. 30; 415(1-2):34-52. Epub 2011 May 27.
Fu K Y, Light A R, Matsushima G K, Maixner W. Microglial reactions after subcutaneous formalin injection into the rat hind paw. Brain Res. 1999 Apr. 17; 825(1-2):59-87.
Garrison C, Dougherty P, Kajander K, Carlton S. Staining of glial fibrillary acidic protein (GFAP) in lumbar spinal cord increases following a sciatic nerve constriction injury. Brain Research (1991) 565:1-7.
Goepfirich A. Mechanism of polymer degradation and erosion. Biomaterials (1996), 17 (2): 103-114.
Goicoechea C, Martin M. Mecanismos periféricos y centrales del dolor. Reumatol Clin (2006), 2 supl 1: S5-S9.
Hains B C. Waxman S G. Activated microglia contributes to the maintenance of chronic pain after spinal cord injury. J Neurosci (2006), 26, 4308-4317.
Hassel B, Paulsen R, Johnsen A, Fonnum F. Selective inhibition of glial cell metabolism in vivo by fluorocitrate. Brain res. (1992), 576: 120-124.
Haydon P G, Glia: listening and talking to the synapse. Nature Reviews Neuroscience (2001), 2: 185-193.
Husmann M, Schenderlein S, Luck M, Lindner H, Kleinebudde P. Polymer erosion in PLGA micropartides produced by phase separation method, int J Pharm (2002), 242(1-2):277-280.
Ilium L, Davis S S, Muller R H, Mak E, West P. The organ distribution and circulation time of intravenously injected colloidal carriers sterically stabilized with a block copolymer-proloxamine 908. Life Sci (1987), 40(4): 367-374.
Inoue A, Ikoma K, Morioka N, Kumagai K, Hashimoto T, Hide I, Nakata Y. Interleukin-1β induces substance P release from primary afferent neurons through the cyclooxygenase-2 system. J Neurochem (1999), 73: 2206-2213.
Ito F, Fujimori H, Makino K. Incorporation of water-soluble drugs in PLGA microspheres. Colloids Surf B Biointerfaces (2007), 54(2):173-178.
Jayo M, Cisneros J. “Guia para el Cuidado y Uso de los Animales de Laboratorio” Institute of Laboratory Animal Resources, commission on Life Sciences National Research Council Edición Mexicana auspiciada por la Academia Nacional De Medicina. 1999. Copyright National Academy Press, Washington, D.C. (1996).
Jiang W, Gupta R K, Deshpande M C, Schwendeman S P. Biodegradable poly(lactic-co-glycolic acid) microparticles for injectable delivery of vaccine antigens. Adv Drug Deliv Rev. 2005 Jan. 10; 57(3):391-410.
Kipp J E. The role of solid nanoparticle thecnology in the parental delivery of poorly water-soluble drugs. Int J Pharmacol (2004), 284:109-122.
Kreutzberg G W. Microglia a sensor for pathological event in the CNS. Trends Neurosci (1996), 19 (8): 312-318.
Kumar N, Ravikumar M N, Domb A J. Biodegradable block copolymers. Adv Drug Deliv Rev (2001), 53(1): 23-44.
Ledeboer A, Liu T, Shumilla J, Mahoney J, Vijay S, Gross M, Vargas J, Suitzbaugh L, Claypool M D, Sanftner L M, Watkins L R, Johnson K W. The glial modulatory drug AV411 attenuates mechanical allodynia in rats models of neuropathic pain. Neuron Glia Biol. (2007), 2: 279-291.
Li M, Rouaud O, Poncelet D. Microencapsulation by solvent evaporation: state of the art for process engineering approaches. Int J Pharm. (2008), 363(1-2):26-39.
Li X, Xu Y, Chen G, Wei P, Ping Q: PLGA nanoparticles for the oral delivery of 5-Fluorouracil using high pressure homogenizationemulsification as the preparation method and in vitro/in vivo studies. Drug Dev Ind Pharm 2008, 34:107-115.
Lipton S A, Singel D J, Staimier J S. Nitric oxide in the central nervous system. Prog Brain res (1994), 103: 359-364.
Liu J, Feng X, Yu M, Xie W, Zhao X, Li W, Guan R, Xu J. Pentoxifylline attenuates development of hiperalgesia in rat model of neuropathic pain. Neurosci lett (2007), 412: 268-272.
Livingston A. Pain and analgesia in non-mammalian species. Thalhammer J G, Short C E. Edición. Proceedings of do Animal Have Pain?, an official satellite symposium of the 9 World Congress on Pain. Vienna, IASP Press 16, (1999).
Mantyh P W, Clohisy D R, Koltzenburg M, Hunt S P. Molecular mechanisms of cancer pain. Nat Rev Cancer (2002), 2(3):201-9.
Meller S T, Dykstra C, Grzybycki D, Murphy S, Gebhart G F. The possible role of glia in nociceptive processing and hyperalgesia in the spinal cord of the rat. Neuropharmacology. 1994 November; 33(11): 1471-8.
Mestre C, Pélissier T, Fialip J, Wilcox G, Eschalier A. A method to perform direct transcutaneous intrathecal injection in rats. J Pharmacol Toxicol Methods (1994), 32(4): 197-200.
Mika J, Osikowicz M, Makuch W, Przew'ocka B. Minocycline and pentoxifylline attenuate allodynia and hyperalgesia and potentiate the effects of morphine in rat and mouse models of neuropathic pain. Eur J Pharmacol (2007), 560: 142-149.
Mika J. Modulation of microglia can attenuate neuropathic pain symptoms and enhance morphine effectiveness. Pharmacol Reports (2008), 60: 297-307.
Miller R F. D-serines a glial modulator of nerve cells. Glia (2004), 47(3): 275-283.
Milligan E D, Watkins L R. Pathological and protective roles of glia in chronic pain. Nature Neuroscience (2009), 10: 23-36.
Nogushi K, R. Dubner, M. A. Ruda. Preproenkephalin mRNA in spinal dorsal horn neurons is induced by peripheral inflammation and is co-localized with Fos and Fos-related proteins. Neuroscience (1992), 46: 561-570.
Paeile J, Bilbeny N. El Dolor, de lo molecular a lo clinico, Eds. 3°, Editorial Mediterráneo, Santiago—Buenos Aires. (2005): 23-41.
Parkinson F E, Paterson A R P, Young J D, Cass C E Inhibitory effects of propentofylline on [3H] adenosine influx. Biochem Pharmacol (1993), 46: 891-896.
Parkinson F E, Fredholm B B. Effects of propentofylline on adenosine A1 and A2 receptors and nitrobenzylthioinosine-sensitive nucleoside transporters: quantitative autoradiographic analysis. Eur J Pharmacol (1991) 202, 361-366.
Platzer C, Fritsch E, Elsner T, Lehmann M H, Volk H D, Prosch S: Cyclic adenosine monophosphate-responsive elements are involved in the transcriptional activation of the human IL-10 gene in monocytic cells. Eur J Immunol (1999), 29: 3098-3104.
Porter J M, Cutler B S, Lee B Y, Reich T, Reichle F A, Scogin J T, Strandness D E. Pentoxifylline efficacy in the treatment of intermittent claudication: multicenter controlled double-blind trial with objective assessment of chronic occlusive arterial disease patients. Am Heart J. 1982 July; 104(1):66-72.
Putney S D. Burke P A, improving protein therapeutics with sustained-release formulations. Nat Biothecnol (1998), 16 (2): 153-157.
Ribeiro-Costa, R. M. et al. In vitro and in vivo properties of usnic acid encapsulated into PLGA-microspheres. Journal of Microencapsulation 21, 371-384 (2004).
Richardson J D, Vasko M R. Cellular mechanisms of neurogenic inflammation. JPET (2002), 302: 839-845.
Rivera A, Cabrero M. Manejo y tratamiento de los animals exoticos. Ediciones Mayo S A, Barcelona, Espa\ddot{n}a, (2008): 100.
Rosea I D, Watari F, Uo M. Microparticle formation and its mechanism in single and double emulsion solvent evaporation. J Control Release. 2004 Sep. 30; 99(2):271-80.
Rother, M., Kittner, B., Rudolphi, K, Robner M, Labs K H. HWA 285 (propentophylline) a new compound for the treatment of both vascular dementia and dementia of the Alzheimer's type. Ann NY Acad Sci (1996), 47: 705-711.
Ruan G, Feng S S, Li Q T. Effects of materials hydrophobicity on physical properties of polymeric microspheres formed by double emulsion process. J Control Release (2002), 84 (3): 151-160.
Scholz J, Woolf C. Can we conquer pain? Nature Neuroscience (2002), 5: 1062-1067.
Schubert P, Rudolphi K. Interfering with the pathologic activation of microglial cells and astrocytes in dementia. Alzheimer Disease and Associates Disorders (1998), 12: S21-8.
Schubert P, Ogata T, Miyazaki H, Marchini C, Ferroni S, Rudolphi K. Pathological immuno-reactions of glial cells in Alzheimer's disease and possible sites of interference. J Neural Transm Suppl. (1998); 54: 167-74.
Schubert P, Merino T, Miyazaki H, Ogata T, Nakamura Y, Marchini C, Ferroni S. Cascading glia reactions: a common pathomechanism and its differentiated control by cyclic nucleotide signaling, Ann N Y Acad Sci. (2000), 903: 24-33,
Shen M R, Chiang P H, Yang R C, Hong C Y, and Chen S S. Pentoxifylline stimulates human sperm motility both in vitro and after oral therapy. Br J Clin Pharmacol. 1991 June; 31(6): 711-714.
Shi, J., Xiao, Z., Votruba, A. R., Vilos, C. & Farokhzad, O. C. Differentially Charged Hollow Core/Shell Lipid-Polymer-Lipid Hybrid Nanoparticles for Small Interfering RNA Delivery. Angew. Chem, int. Ed. 50, 7027-7031 (2011).
Shinoda I, Furukawa Y, Furukawa S. Stimulation of nerve growth factor synthesis/secretion by propentofylline in cultured mouse astroglial cells. Biochem Pharmacol (1990), 39: 1813-1816.
Si Q, Nakamura Y, Ogata T, Kataoka K, Schubert P. Differential regulation of microglial activation by propentofylline via cAMP signaling. Brain Res (1998), 23: 97-104.
Patent Application WO9702022, Biocompatible and biodegradable nanoparticles designed for proteinaceous drugs absorption and delivery; Univ liege; Grandfils Christian; Jerome Robert. Fecha de publicación 2 Feb. 1996.
Patent Application WO2008054042, Bio-degradable nanoparticles of poly(DL-lactide-co-glycolide) encapsulating ciprofloxacin HCl having an extended-release property, and manufacturing method thereof; Nah Jae Woon; Jung Teok Rae; Jeong Young. Fecha de publicación 5 May 2007,
Patent Application WO2007127363, Coatings containing multiple drugs; Micell Technologies Inc; Deyoung James; Taylor Doug; Mcclain Jim; Smoke Clint. Fecha de publicación 28 Apr. 2007.
Patent Application WO2009147372, Drug delivery composition; Univ Belfast; Mccoet alin Peter; Gorman Sean Patrick; Jones David Simon. Fecha de publicación 28 May 2009.
Patent Application WO2010009335, Drug delivery medical device; Micell Technologies Inc; Mcclain James B; Taylor Douglas; Neet John, Fecha de publicación 16 Jul. 2009.
Patent Application WO2008128123, Formulation of active agent loaded activated plga nanoparticles for targeted cancer nano-therapeutics; Univ North Texas; Braden Arthur R C; Vishwanatha Jamboor K; Kafka Erica; Fecha de publicación 11 Apr. 2008.
Patent Application WO2010111238, improved biodegradable polymers; Micell Technologies Inc; Taylor Douglas; Mcclain James B; Schmitt Edward. Fecha de publicación 23 Mar. 2010.
Patent Application WO2005084710, Nanocell drug delivery system; Massachusetts Inst Technology; Sengupta Shiladitya; Zhao Ganlin; Capila Ishan; Eavarone David. Fecha de publicación 2 Mar. 2005.
Sweitzer S M, Colburn R, Rutkdwski M., DeLeo J. Acute peripheral inflammation induces moderate glial activation and spinal IL-1β expression that correlates with pain behavior in the rat. Brain Res (1999), 829:209-221.
Sweitzer S M, Schubert P, DeLeo J. A., Propentofylline, a glial modulating agent, exhibits antiallodynic properties in a rat model of neuropathic pain, J Pharmacol Exp Ther 297 (2001), 1210-1217.
Sweitzer S M, DeLeo J. Propentofylline: glial modulation, neuroprotection and alleviation of chronic pain. Handb Exp Pharmacol (2011), 200: 235 -250.
Tawfik V L, Regan M R, Haenggeli C, LaCroix-Fralish M L, Nutile—McMenemy N, Perez N, Rothstein J D, DeLeo J A. Propentofylline induced astrocyte modulation leads to alterations in glial glutamate promoter activation following spinal nerve transaction. Neuroscience (2008), 152: 1086-1092.
Terman G, Bonica J. Spinal mechanisms and their modulation. En: Loesser J, Butler S, Chapman C R, Turk D C, editores. Bonica's Management of Pain. 3rd ed. Philadelphia: Lippincott Williams & Wiikins; (2001), 73-152.
Tikka T, Fiebich B, Goldsyein G, Keinanen R, Koistinaho J. Minocycline, a tetracycline derivate, is neuroprotective against excitotoxicity by inhibition activation and proliferation of microglia. J Neurosci (2001), 21: 2580-2588.
Turk D C, Okifuji A. Pain terms and taxonomies of pain. In: Loeser J D, Butler S H, Chapman C R, Turk D C, editors. Cole: Pain Management: pp. 23-30 Bonica's management of pain. 3rd ed. Philadelphia: Lippincott Williams & Wilkins; 2001:17-25
Waeckerle-Men Y, Groettrup M. PLGA microspheres for improved antigen delivery to dendritic cells as cellular vaccines. Adv Drug Deliv Rev (2005), 57(3): 475-482.
Watkins L R, Milligan E D, Maier S F. Glial activation: a driving force for pathological pain. TINS. (2001), 24: 450-455.
Watkins L R, Maier S F. Beyond neurons: evidence that immune and glial cells contributes to pathological pain states. Physiol Rev (2002), 82 (4): 981-1011.
Woolf C, Salter M. Neuronal plasticity: increasing the gain in pain. Science (2000), 288: 1765-1788.

1. A method of synthesizing nanoparticles of poly (lactic-glycolic acid) (PLGA) with pentoxifylline encapsulated, comprising the steps of a) dissolving PLGA polymer in a solvent; b) dissolving in purified water pentoxifylline; c) addition of pentoxifylline dissolved in step b) the dissolved PLGA polymer in step a) previously cooled; d) emulsifying the mixture of step e); e) adding a solution of an emulsifying agent in purified water; f) homogenizing the mixture of step e); g) diluting me homogenization of step f) in purified water; h) evaporation of the solvent in which the PLGA polymer is dissolved in step a); i) washing the nanoparticles with purified water; j) collection of nanoparticles in solution.

2. Synthesis Method according to claim 1, wherein the PLGA polymer used in step a) has a ratio range of polylactic acid to polyglycolide acid ranging from 10% to 90% polylactic acid with polyglycolie acid up to 90% of polylactic acid with 10% polyglycolie acid or any combination that fails within these ranges.

3. Synthesis Method according to claim 1, wherein the PLGA polymer used in step a) has a ratio of 50% polylactic acid and 50% polyglycolie acid,

4. Synthesis Method according to claim 1, wherein the solvent used in step a) is dichloromethane or chloroform.

5. The method of synthesis according to claim 1, wherein the ratio of polymer to PLGA used pentoxifylline from 0.01% 0.0003% PLGA and pentoxifylline, up to 9% and 0.3% pentoxifylline PLGA or any combination that falls within these ranges.

6. The method of synthesis according to claim 1, wherein the ratio of polymer to PLGA is used pentoxifylline PLGA 0.3% and 0.01% pentoxifylline.

7. Method of synthesis according to claim 1, wherein step d) emulsifying the mixture of step c) is performed using a sonicator.

8. The method of synthesis according to claim 7 wherein the emulsion in the sonicator is performed at a frequency of 10 to 30 kHz, at a power of 90-170 watts for 40 to 80 seconds.

9. The method of synthesis according to claim 7 wherein the emulsion in the sonicator is performed at a frequency of 20 kHz, at a power of 130 watts for 60 seconds.

10. A method of synthesis according to claim L wherein the emulsifying agent used in the solution of step e) is polyvinyl alcohol (PVA), polyethylene glycol or its derivatives, anionic or cationic emulsifiers and pharmaceutical use or any combination they.

11. A method of synthesis according to claim 10, when PVA is used in step e), it is hydrolyzed in 80 to 95%,

12. The method of synthesis according to claim 10, when PVA is used in step e), it is 87-89% hydrolyzed.

13. The method of synthesis according to claim 10, when PVA is used in step e), the PVA solution in purified water comprises 0.1 to 5% w/v PVA.

14. The method of synthesis according to claim 10, when PVA is used in step e), the PVA solution in purified water comprising 0.5% w/v PVA.

15. A method of synthesis according to claim 1, wherein the homogenization of step f) is performed using a sonicator, by vortexing, by an ultra-turrax homogenizer using high pressure homogenizers or by any or any combination from them.

16. The method of synthesis according to claim 15 wherein the homogenization of step f) is performed using a sonicator with a frequency of 10 to 30 kHz, at a power of 90-170 watts, for 5 to 70 seconds.

17. A method of synthesis according to claim 15, wherein the homogenization in the sonicator is performed at a frequency of 20 kHz, at a power of 130 watts for 15 seconds and then repeat for 40 seconds.

18. The method of synthesis according to claim 1 wherein the evaporation, of the solvent in step h) is carried out by stirring the solution, gas streams as nitrogen or oxygen, heat, freeze drying or any combination thereof.

19. A method of synthesis according to claim 18, wherein evaporation of the solvent in step h) is effected by moderate orbital shaking (70 to 170 rpm) at room temperature for 5 to 20 hours.

20. The method of synthesis according to claim 1 wherein the collection of the nanoparticles in solution from step j) is performed by filtration, centrifugation and filtration, differential centrifugation, gradient centrifugation or any combination thereof.

21. A method of synthesis according to claim 20 wherein the nanoparticles are collected by centrifugation and filtration through a filter with 80 to 120 KDa cut and centrifuging at 3500 to 5500G for the time necessary to obtain a solution with nanoparticles.

22. A method of synthesis according to claim 21, wherein the filtration for collection of nanoparticles is performed by a 100 kDa cut filter and centrifuging at 4500G for the time necessary to obtain a solution with nanoparticles.

23. A pharmaceutical formulation for preventing and alleviating chronic pain, comprising nanoparticles of poly(lactic-glycolic acid) (PLGA) with pentoxifylline encapsulated in a form which allows the administration of a single dose.

24. Pharmaceutical formulation according to claim 23 wherein the nanoparticles of poly(lactic-glycolic acid) (PLGA) have a range of polylactide acid to polyglycolic acid ranging from 10% of polylaetie acid with 90% acid polyglycolic, up to 90% of polylaetic acid with 10% polyglycolic acid or any combination that fails within these ranges.

25. Pharmaceutical formulation according to claim 23 wherein the nanoparticles of poly(lactic-glycolic acid) (PLGA) with a ratio of 50% polylaetie acid and 50% polyglycolic acid.

26. Pharmaceutical formulation according to claim 23, wherein the nanoparticles loaded PLGA pentoxifylline have a uniform size between 1.50 and 410 inn.

27. Pharmaceutical formulation according to claim 23, wherein the nanoparticles loaded P1..GA pentoxifylline have a homogeneous size of 250 nm.

28. Pharmaceutical formulation according to claim 23, wherein the nanoparticles loaded PLGA pentoxifylline are spherical with a smooth surface and hydrophilic, polymeric shell and a central cavity that is encapsulated pentoxifylline.

29. Pharmaceutical, formulation according to claim 23, wherein the nanoparticles loaded PLGA pentoxifylline have a negative surface charge with a value between −24 and −13 mV.

30. Pharmaceutical formulation according to claim 23, wherein PLGA nanoparticles loaded with PTX possess a negative surface charge of −18.5 mV value.

31. Pharmaceutical formulation according to claim 23, wherein the proportion of pentoxifylline encapsulated in PLGA nanoparticles is between 1 .mu.g in 10 .mu.i of pentoxifylline nanoparticle solution until 3 μg of pentoxifylline in 10 .mu.i of solution of nanoparticles or any ratio that falls within these ranges.

32. Pharmaceutical formulation according to claim 23, wherein the proportion of pentoxifylline encapsulated in PLGA nanoparticles is 2 μg of pentoxifylline in 10 .mu.l of solution of nanoparticles.

33. Pharmaceutical formulation according to claim 23, wherein the loaded PLGA nanoparticles are stable pentoxifylline and no significant change in size when exposed to low temperature or lyophilization.

34. Pharmaceutical formulation according to 23, further comprising any additive, agent or pharmaceutically suitable adjuvant.

35. Pharmaceutical formulation according to claim 23, wherein the single dose administration is intrathecal, intravenous, or intramuscular,

36. Pharmaceutical formulation according to claim 23, wherein the single dose of pharmaceutical formulation, the controlled and sustained loaded PLGA nanoparticles pentoxifylline with pharmacological effect of the present invention slowly release is in the range of 0.001 to 0.1 mg of pentoxifylline encapsulated/kilo of body weight of subject suffering or known to suffer chronic pain.

37. Pharmaceutical formulation according to claim 23, wherein the single dose of controlled and sustained-release pharmaceutical formulation of loaded PLGA nanoparticles pentoxifylline with pharmacological effect of the present invention is encapsulated 0.01 mg/kilo of body weight of the subject suffering or known to suffer chronic pain pentoxifylline.

38. Method of alleviating or preventing chronic pain which comprises administering a dose comprising nanoparticles loaded PLGA pentoxifylline to a patient in need thereof.

39. Method according to claim 38, wherein the nanoparticles of poly(lactic-glycolic acid) (PLGA) have a range of ratio of polylactic acid to polyglycolic acid ranging from 10% to 90% polylactic acid with polyglycolic acid , up to 90% of polylactic acid with 10% polyglycolic acid or any combination that fails within these ranges.

40. Method according to claim 38, wherein the nanoparticles of poly (lactic-glycolic acid) (PLGA) with a ratio of 50% polylactic acid and 50% polyglycolic acid.

41. Method according to claim 38, wherein the nanoparticles loaded PLGA pentoxifylline have a homogeneous size of between 150 and 410 nm.

42. Method according to claim 38, wherein the nanoparticles loaded PLGA pentoxifylline have a homogeneous size of 250 nm.

43. Method according to claim 38, wherein the nanoparticles loaded PLGA pentoxifylline are spherical, with a smooth and hydrophilic surface, a polymeric shell and a central cavity that is encapsulated pentoxifylline,

44. Method according to claim 38, wherein the nanoparticles loaded PLGA pentoxifylline have a negative surface charge with a value between −24 and −13 mV.

45. Method according to claim 38, wherein PLGA nanoparticles loaded with PTX possess a negative surface charge of −18.5 mV value.

46. Method according to claim 38, wherein the proportion of pentoxifylline encapsulated in PLGA nanoparticles is between 1 .mu.g in 10 .mu.l of pentoxifylline nanoparticle solution until 3 μg of pentoxifylline in 10 .mu.l of solution of nanoparticles or any proportion it is within these ranges.

47. Method according to claim 38, wherein the proportion of pentoxifylline encapsulated in PLGA nanoparticles is pentoxifylline 2 μg in 10 .mu.l of solution of nanoparticles.

48. Method according to claim 38, wherein the nanoparticles loaded PLGA pentoxifylline are stable and no significant change In size when exposed to low temperature or lyophilization.

49. Method according to claim 38, wherein the pharmaceutical formulation further comprises any additive, agent or pharmaceutically suitable adjuvant.

50. Method according to claim 38, wherein the single dose administration is intrathecal, intravenous, or intramuscular.

51. Method according to claim 38, wherein the single dose of pharmaceutical formulation of slow release, controlled and sustained PLGA nanoparticles loaded with pentoxifylline pharmacological effect of the present invention is in the range of 0.001 to 0.1 mg of pentoxifylline encapsulated / kilo of body weight of the subject suffering or known to suffer chronic pain.

52. Method according to claim 38, wherein the single dose of pharmaceutical formulation of slow release, controlled and sustained PLGA nanoparticles loaded with pentoxifylline pharmacological effect of the present invention is 0.01 mg of pentoxifylline encapsulated / kilo of body weight of the subject suffering or known to suffer chronic pain.
